(19) United States
(12) Patent Application Publication
(10) Pub. No.: US 2011/0182866 A1
(43) Pub. Date: Jul. 28, 2011

(54) ISOLATION OF STEM CELL PRECURSORS AND EXPANSION IN NON-ADHERENT CONDITIONS

(75) Inventor: Ian K. Mcniece, Coral Gables, FL (US)

(73) Assignee: UNIVERSITY OF MIAMI, Miami, FL (US)

(21) Appl. No.: 12/992,627

(22) PCT Filed: May 14, 2009
(86) PCT No.: PCT/US2009/043885

§ 371 Date: Apr. 8, 2011

Related U.S. Application Data
(60) Provisional application No. 61/053,462, filed on May 15, 2008.

(51) Int. Cl. A61K 35/28; C12N 13/00; C12N 5/071; A61K 35/12; C12M 3/00; A61P 9/00

(52) U.S. Cl. 424/93.7; 435/173.9; 435/325; 435/289.1

(57) ABSTRACT

Stem cells and compositions thereof are isolated, cultured and expanded. Culture conditions and methods of culturing the isolated stem cells provide non-adherent stem cells which are prophylactically and therapeutically more effective in patients, diagnostics, screening assays and other stem cell uses.

### CROSS-REFERENCE TO RELATED APPLICATIONS

The present application claims the priority of U.S. provisional patent application No. 61/053,462 filed May 15, 2008, and is incorporated herein by reference in its entirety.

### FIELD OF THE INVENTION

The invention relates to stem cell isolation, expansion, culturing and uses thereof.

### BACKGROUND

The use of bone marrow derived mesenchymal stem cells (MSC) has been proposed for a number of regenerative therapies including repair of myocardial tissue. These studies have utilized MSC grown as adherent cells in plastic tissue culture flasks and trypsinized for harvest and infusion. There is a significant probability for contamination in such open systems and the use of trypsin for passage of the mesenchymal stem cells may affect surface proteins on the mesenchymal stem cells or potentially induce transformation. In addition, transplantation of plastic adherent mesenchymal stem cells (PA-MSC) into tissues, such as the heart, may result in failure of the plastic adherent mesenchymal stem cells to integrate into the tissue due to the lack of an adherent substrate for the plastic adherent mesenchymal stem cells. The development of conditions that will isolate and maintain stem cells in vitro for the extended periods of time required for the procedures involved in stem cell transplantation, tissue repair, regeneration, gene therapy, identification of growth factors, thorough characterization of cell morphologies and the like, has also presented a unique set of obstacles. To date, successful in vitro stem cell cultures have depended on the ability of the laboratory worker to mimic the conditions which are believed to be responsible for maintaining stem cells in vivo.

### SUMMARY

This Summary is provided to present a summary of the invention to briefly indicate the nature and substance of the invention. It is submitted with the understanding that it will not be used to interpret or limit the scope or meaning of the claims.

Culture conditions for the generation of non adherent stem cells provide a closed culture system with a decreased probability of contamination and allow for passage without the use of enzyme treatment such as trypsin.

In a preferred embodiment, a method of isolating non-adherent stem cells comprises obtaining a biological sample from an animal; isolating bone marrow from the biological sample; isolating and culturing cells obtained from the bone marrow; contacting the cells with antibody and separating the cells to isolate stem cells; and, isolating non-adherent stem cells.

In another preferred embodiment, the antibody is directed to a cell surface marker comprising at least one of: CD271, CXCR4, CD 133, SCA-1, Tra-1-60, CD 44, CD 73, CD 90, CD 105 or Stro-1.

In another preferred embodiment, the non-adherent stem cells are isolated by phenotype CD271⁺, CD105⁻.

In another preferred embodiment, the stem cells are separated by magnetic or cell sorting means.

In another preferred embodiment, the cells arc cultured in a pliable tissue culture container and mechanical forces are applied to prevent adherence of the cells in the tissue culture container.

In another preferred embodiment, the non-adherent stem cells are precursor mesenchymal stem cells.

In another preferred embodiment, the animal is a human subject.

In another preferred embodiment, a method of repairing and regenerating tissue in an animal comprises isolating stem cells from bone marrow of an animal; isolating, culturing, and expanding non-adherent stem cells; and, transferring the non-adherent stem cells into the animal.

In another preferred embodiment, the isolated stem cells are optionally cultured in tissue culture comprising differentiation or growth factors.

In another preferred embodiment, the stem cell recipient animal is also the donor of the bone marrow.

In another preferred embodiment, the stem cells are obtained from allogeneic, autologous or syngeneic sources.

In another preferred embodiment, the stem cells are non-adherent mesenchymal stem cells.

In another preferred embodiment, the stem cells are transplanted into cardiac tissues.

In another preferred embodiment, a method of repairing and regenerating heart tissue in a patient comprises isolating stem cells from bone marrow of a donor; isolating, culturing, and expanding non-adherent stem cells; and, transferring the non-adherent stem cells into the patient.

In another preferred embodiment, the isolated stem cells are optionally cultured in tissue culture comprise differentiation or growth factors.

In another preferred embodiment, the stem cell recipient patient is also the donor of the bone marrow.

In another preferred embodiment, the stem cells are obtained from allogeneic, autologous or syngeneic sources.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a scan of a photograph showing a cytospin preparation of BM derived CD271⁺ cells.

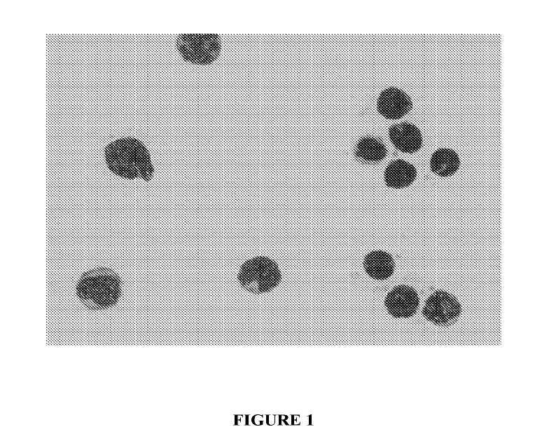

FIGS. 2A-2C are scans of photographs showing the generation of non-adherent mesenchymal stem cells (NA-MSC) from CD271⁺ cells. The figures show the proliferation of CD271⁺ cells from 7 (FIG. 2A), 14 days (FIG. 2B) and 21 days (FIG. 2C).

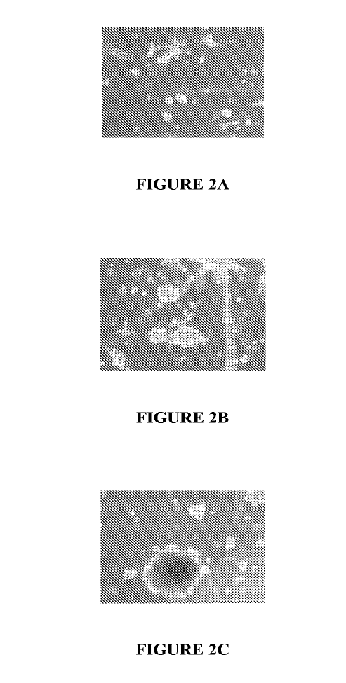

FIG. 3 is a scan of a photograph showing culture of NA-MSC in plastic tissue flasks which demonstrates typical MSC morphology.

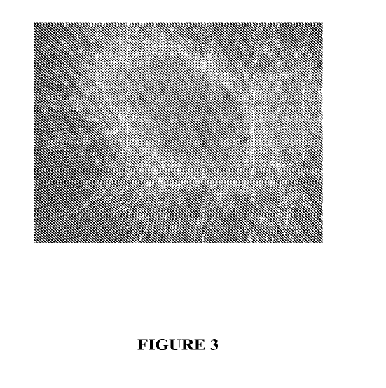

FIG. 4 is a flow cytometry scan showing CD105-FITC staining of non-adherent mesenchymal stem cells (NA-MSC).

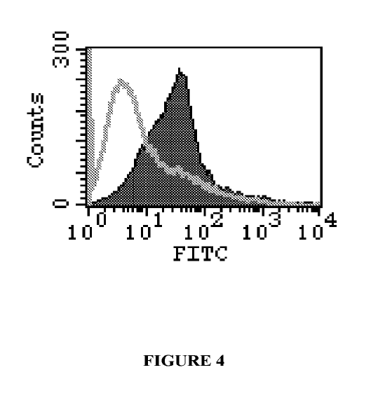

FIG. 5 is a scan of a photograph showing the differentiation of non-adherent mesenchymal stem cells (NA-MSC).

FIG. 6 is a scan of several photographs showing the expression of cardiogenic markers after culture of non-adherent mesenchymal stem cells (NA-MSC) for 3 weeks in a cocktail of growth factors shown to stimulate cardiac differentiation.

FIG. 7 is a graph showing the ejection fraction of mice at baseline and post infarction (1, 2 and 4 weeks).

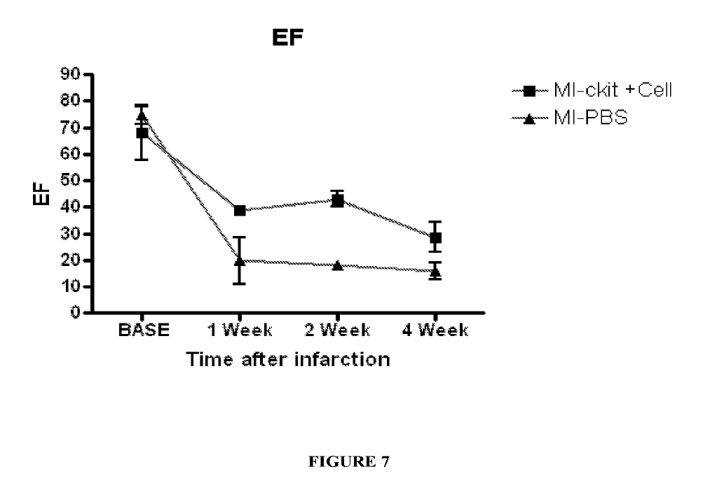

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Mesenchymal stem cells (MSC) were grown in a closed bag system which enabled passage of the cells without the use of trypsin. The isolated non-adherent mesenchymal stem cells (NA-MSC) have a different phenotype to plastic adherent mesenchymal stem cells (PA-MSC) but maintained the ability to adhere to plastic. The non-adherent mesenchymal stem cells have a greater capacity to integrate into a tissue environment and continue to proliferate, resulting in regeneration of damaged tissue.

### Definitions

Prior to setting forth the invention, the following definitions are provided:

As used herein, the singular forms “a”, “an” and “the” include plural referents unless the context clearly dictates otherwise.

“Isolating” a stem cell refers to the process of removing a stem cell from a tissue sample and separating away other cells which are not stem cells of the tissue. An isolated stem cell will be generally free from contamination by other cell types and will generally have the capability of propagation and differentiation to produce mature cells of the tissue from which it was isolated. However, when dealing with a collection of stem cells, e.g., a culture of stem cells, it is understood that it is practically impossible to obtain a collection of stem cells which is 100% pure. Therefore, an isolated stem cell can exist in the presence of a small fraction of other cell types which do not interfere with the utilization of the stem cell for analysis or production of other, differentiated cell types. Isolated stem cells will generally be at least 30%, 40%, 50%, 60%, 70%, 80%, 85%, 90%, 95%, 98%, or 99% pure. Preferably, isolated stem cells according to the invention will be at least 98% or at least 99% pure.

A stem cell is “expanded” when it is propagated in culture and gives rise by cell division to other stem cells and/or progenitor cells. Expansion of stem cells may occur spontaneously as stem cells proliferate in a culture or it may require certain growth conditions, such as a minimum cell density, cell confluence on the culture vessel surface, or the addition of chemical factors such as growth factors, differentiation factors, or signaling factors.

A stem cell, progenitor cell, or differentiated cell is “transplanted” or “introduced” into a mammal when it is transferred from a culture vessel into a patient. Transplantation, as used herein, can include the steps of isolating a stem cell according to the invention and transferring the stem cell into a mammal or a patient. Transplantation can involve transferring a stem cell into a mammal or a patient by injection of a cell suspension into the mammal or patient, surgical implantation of a cell mass into a tissue or organ of the mammal or patient, or perfusion of a tissue or organ with a cell suspension. The route of transferring the stem cell or transplantation, will be determined by the need for the cell to reside in a particular tissue or organ and by the ability of the cell to find and be retained by the desired target tissue or organ. In the case where a transplanted cell is to reside in a particular location, it can be surgically placed into a tissue or organ or injected into the bloodstream if the cell has the capability to migrate to the desired target organ.

An “immunosuppressive agent” is any agent that prevents, delays the occurrence of or reduces the intensity of an immune reaction against a foreign cell in a host, particularly a transplanted cell. Preferred are immunosuppressive agents which suppress cell-mediated immune responses against cells identified by the immune system as non-self. Examples of immunosuppressive agents include but are not limited to cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art.

A “differentiation factor” is any agent that causes a stem cell or progenitor cell to differentiate into another cell type. Differentiation is usually accomplished by altering the expression of one or more genes of the stem cell or progenitor cell and results in the cell altering its structure and function.

A “signaling factor” as used herein is an agent secreted by a cell which has an effect on the same or different cells. For example, a signaling factor can inhibit or induce the growth, proliferation, or differentiation of itself, neighboring cells, or cells at distant locations in the organism. Signaling factors can, for example, transmit positional information in a tissue, mediate pattern formation, or affect the size, shape and function of various anatomical structures.

The terms, “patient”, “subject” or “animal” are used interchangeably and refer to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, a mammal refers to any mammal including but not limited to human, mouse, rat, sheep, monkey, goat, rabbit, hamster, horse, cow or pig.

As used herein, “allogeneic” refers to genetically different members of the same species.

As used herein, “isogeneic” refers to an identical genetic constitution.

As used herein, “xenogeneic” refers to members of a different species.

As used herein, “syngeneic” refers to a genetically compatible constitution, allowing for the transplantation of tissue without provoking an immune response.

As used herein, “culturing” refers to propagating or nurturing a cell, collection of cells, tissue, or organ, by incubating for a period of time in an environment and under conditions which support cell viability or propagation. Culturing can include one or more of the steps of expanding and proliferating a cell, collection of cells, tissue, or organ according to the invention.

“Transplantation” as used herein, can include the steps of isolating a stem cell according to the invention, and culturing and transferring the stem cell into a mammal or a patient. Transplantation, as used herein, can include the steps of isolating a stem cell according to the invention, differentiating the stem cell, and transferring the stem cell into a mammal or a patient. Transplantation, as used herein, can include the steps of isolating a stem cell according to the invention, differentiating and expanding the stem cell and transferring the stem cell into a mammal or a patient.

### Stem Cell Isolation and Expansion

The culture system provides stem cells which are appropriate for use in transplantations, treatments and other uses, and lack the problems associated with current stem cell isolation and culture technologies.

In a preferred embodiment, a stem cell is isolated from bone marrow of an animal, preferably a mammal. In accordance with the invention, stem cells from a patient or animal are harvested, sorted, purified and identified. A purified or isolated population of stem cells contains a significantly higher proportion of stem cells than the crude population of cells from which the stem cells are isolated. For example, the purification procedure should lead at least to a five fold increase, preferably at least a ten fold increase, more preferably at least a fifteen fold increase, most preferably at least a twenty fold increase, and optimally at least a twenty-five fold increase in stem cells with respect to the total population. The purified population of stem cells should include at least 15%, preferably at least 20%, more preferably at least 25%, most preferably at least 35%, and optimally at least 50% of stem cells.

In another preferred embodiment, isolated stem cells are CD271⁺, CD105⁻. These same cells can become CD105⁻ cells once they are cultured.

Other stem cell specific marker molecules include, but not limited to: CXCR4, CD 133, SCA-1, Tra-1-60, CD 44, CD 73, CD 90, CD 105 and Stro-1.

In another preferred embodiment, a stem cell is cultured in a culture container that is supple and can be manipulated, to which an external mechanical force can be applied.

In one embodiment, the culture container is coated with a surface that decreases the probability of cells attaching Examples include polytetrafluoroethylene or polytetrafluoroethene (PTFE) (TEFLON), PFA (perfluoroalkoxy polymer resin), FEP (fluorinated ethylene-propylene) coated tissue culture bags or plates.

In another preferred embodiment, the cell culturing chamber is made of a material to which the stem cells cannot or do not adhere to. This can include any, plastics, polymers, glass, silicon based compounds or any other material that is deemed to prevent or is not conducive to adherence of the cells. In addition, the cell culture chamber can be pre-treated or coated with any one or more of nucleic acids, peptides, polypeptides, enzymes, antibodies, organic or inorganic molecules that prevent a cell from binding to the surface. For example, adhesion molecules include but are not limited to Ig superfamily CAM's, integrins, cadherins and selectins and their neutralizing antibodies. The chamber can be pretreated or the tissue culture medium can comprise antibodies to prevent adherence of cells to the culturing surface.

The cells can be treated for example, with oligonucleotides, that encode or block by antisense, ribozyme activity, or RNA interference transcription factors that are involved in regulating gene expression of extracellular matrix components, or other molecular activities that regulate differentiation.

Extracellular matrix components include but are not limited to Keratin Sulphate Proteoglycan, Laminin, Chondroitin Sulphate A, SPARC, beta amyloid precursor protein, beta amyloid, presenilin 1,2, apolipoprotein E, thrombospondin-1,2, Heparan Sulphate, Heparan sulphate proteoglycan, Matrigel, Aggregan, Biglycan. Poly-L-Ornithine, the collagen family including but not limited to Collagen I-IV, Poly-D-Lysine, Ecistatin (Viper Venom), Flavoridin (Viper Venom), Kistrin (Viper Venom), Vitronectin, Supeffibronectin. Fibronectin Adhesion-Promoting peptide, Fibronectin Fragment Fibronectin Fragment-30KDA, Fibronectin-Like Polymer, Fibronectin Fragment 45KDA, Fibronectin Fragment 70KDA, Asialoganglioside-GM, Disialoganglioside-GOLA-, Monosialo Ganglioside-GM₁, Monosialoganglioside-GM₂, Monosialoganglioside-GM₃, Methylcellulose, Keratin Sulphate Proteoglycam, Laminin and Chondroitin Sulphate A.

The cell culturing chamber can be a cell culture bag so that it is soft and can be squeezed so as to agitate the cells or the chamber can be a typical culture dish and the cells are cultured and stirrer with, for example, a magnetic stirrer.

In another preferred embodiment, the cells can be grown in conditions where the tissue culture containers are gently rocked, rotated, swirled, and moved in a circular fashion and the like. Tissue culture containers can be any types that are available to one of skill in the art. In some preferred embodiments, tissue culture bags are preferred so that the bags can be manipulated or massaged. The Examples section which follows provides a detailed description of the preferred methods.

Although, preferred methods of isolating and purifying stem cells are described, other methods known in the art may be used. For example, various techniques may be employed to separate the cells by initially removing cells of dedicated lineage. Monoclonal antibodies are particularly useful for identifying markers associated with particular cell lineages and/or stages of differentiation.

If desired, a large proportion of terminally differentiated cells may be removed by initially using a “relatively crude” separation. For example, magnetic bead separations may be used initially to remove large numbers of lineage committed cells. Desirably, at least about 80%, usually at least 70% of the total hematopoietic cells will be removed.

Procedures for separation may include but are not limited to, magnetic separation, using antibody-coated magnetic beads, affinity chromatography, cytotoxic agents joined to a monoclonal antibody or used in conjunction with a monoclonal antibody, including but not limited to, complement and cytotoxins, and “panning” with antibody attached to a solid matrix, e.g., plate, elutriation or any other convenient technique.

Techniques providing accurate separation include but are not limited to, flow cytometry, which can have varying degrees of sophistication, e.g., a plurality of color channels, low angle and obtuse light scattering detecting channels, impedance channels, etc.

In a preferred embodiment, the stem cells are mesenchymal stem cell precursors, however, any stem cell can be used. Non-limiting examples of stem cells, which can be used according to this aspect of the present invention, are hematopoietic stem cells (HSCs) and mesenchymal stem cells (MSCs) obtained from bone marrow tissue of an individual at any age or from cord blood of a newborn individual, embryonic stem (ES) cells obtained from the embryonic tissue formed after gestation (e.g., blastocyst), or embryonic germ (EG) cells obtained from the genital tissue of a fetus any time during gestation, preferably before 10 weeks of gestation.

HSCs—Hematopoietic stem cells (HSCs) are the formative pluripotential blast cells found inter alia in fetal liver, umbilical cord blood, bone marrow and peripheral blood which are capable of differentiating into any of the specific types of hematopoietic or blood cells, such as erythrocytes, lymphocytes, macrophages and megakaryocytes. Typically, within the bone marrow, HSCs reside in niches that support all the requisite factors and adhesive properties to maintain their ability and produce an appropriate balanced output of mature progeny over the life time of the organism [Whetton (1999) Trends Cell Biol 9:233-238; Weissman (2000) Cell 100:157-168; Jankowska-Wieczorek (2001) Stem Cells 19:99-107; Chan (2001) Br. J. Haematol. 112:541-557].

HSCs according to this aspect of the present invention are preferably CD34⁺ cells and more preferably CD34⁺/CD38⁻/low cells, which are a more primitive stem cell population and are therefore less lineage-restricted, and were shown to be the major long-term BM repopulating cells.

MSCs—Mesenchymal stem cells are the formative pluripotential blast cells found inter alia in bone marrow, blood, dermis and periosteum that are capable of differentiating into more than one specific type of mesenchymal or connective tissue (i.e. the tissues of the body that support the specialized elements; e.g. adipose, osseous, stroma, cartilaginous, elastic and fibrous connective tissues) depending upon various influences from bioactive factors, such as cytokines

Approximately, 30% of human marrow aspirate cells adhering to plastic are considered as MSCs. These cells can be expanded in vitro and then induced to differentiate. The fact that adult MSCs can be expanded in vitro and stimulated to form bone, cartilage, tendon, muscle or fat cells render them attractive for tissue engineering and gene therapy strategies. In vivo assays have been developed to assay MSC function. MSCs injected into the circulation can integrate into a number of tissues described hereinabove. Specifically, skeletal and cardiac muscle can be induced by exposure to 5-azacytidine and neuronal differentiation of rat and human MSCs in culture can be induced by exposure to β-mercaptoethanol, DMSO or butylated hydroxyanisole [Woodbury (2000) J. Neurosci. Res. 61:364-370]. Furthermore, MSC-derived cells are seen to integrate deep into brain after peripheral injection as well as after direct injection of human MSCs into rat brain; they migrate along pathways used during migration of neural stem cells developmentally, become distributed widely and start lose markers of HSC specialization [Azizi (1998) Proc. Natl. Acad. Sci. USA 95:3908-3913]. Methods for promoting mesenchymal stem and lineage-specific cell proliferation are disclosed in U.S. Pat. No. 6,248,587.

Epitopes on the surface of the human mesenchymal stem cells (hMSCs) such as SH2, SH3 and SH4 described in U.S. Pat. No. 5,486,359 can be used as reagents to screen and capture mesenchymal stem cell population from a heterogeneous cell population, such as exists, for example, in bone marrow. Precursor mesenchymal stem cells which are positive for CD45 are preferably used according to this aspect of the present invention, since these precursor mesenchymal stem cells can differentiate into the various mesenchymal lineages.

Preferred stem cells according to this aspect of the present invention are human stem cells.

Adult stem cells can be obtained using a surgical procedure such as bone marrow aspiration or can be harvested using commercial systems such as those available from Nexell Therapeutics Inc. Irvine, Calif., USA.

Stem cells utilized by the present invention can also be collected (i.e., harvested) using a stem cell mobilization procedure, which utilizes chemotherapy or cytokine stimulation to release of HSCs into circulation of subjects. Stem cells are preferably retrieved using this procedure since mobilization is known to yield more HSCs and progenitor cells than bone marrow surgery.

Stem cell mobilization can be induced by a number of molecules. Examples include but are not limited to cytokines such as, granulocyte colony-stimulating factor (G-CSF), granulocyte-macrophage colony-stimulating factor (GM-CSF), interleukin (IL)-7, IL-3, IL-12, stem cell factor (SCF), and flt-3 ligand; chemokines like IL-8, Mip-1α, Groβ, or SDF-1; and the chemotherapeutic agents cyclophosphamide (Cy) and paclitaxel. It will be appreciated that these molecules differ in kinetics and efficacy, however, according to presently known embodiments G-CSF is preferably used alone or in combination such as with cyclophosphamide to mobilize the stem cells. Typically, G-CSF is administered daily at a dose of 5-10 μg/kg for 5-10 days. Methods of mobilizing stem cells are disclosed in U.S. Pat. Nos. 6,447,766 and 6,162,427.

Human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; Gardner et al., [Fertil. Steril. 69: 84, 1998].

It will be appreciated that commercially available stem cells can be also be used according to this aspect of the present invention. Human ES cells can be purchased from the NIH human embryonic stem cells registry (escr.nih.gov). Non-limiting examples of commercially available embryonic stem cell lines are BG01, BG02, BG03, BG04, CY12, CY30, CY92, CY10, TE03, TE32.

Human EG cells can be retrieved from the primordial germ cells obtained from human fetuses of about 8-11 weeks of gestation using laboratory techniques known to anyone skilled in the arts. The genital ridges are dissociated and cut into small chunks, which are thereafter disaggregated into cells by mechanical dissociation. The EG cells are then grown in using the methods described herein.

It will be appreciated that enrichment of stem cell population exhibiting pluripotency may be preferably effected. Thus, for example, CD127⁺ cells can be concentrated using affinity columns or FACS.

Culturing of stem cells under proliferative conditions may also be effected in cases where stem cell numbers are too low for use in treatment. Culturing of stem cells is described in U.S. Pat. Nos. 6,511,958, 6,436,704, 6,280,718, 6,258,597, 6,184,035, 6,132,708 and 5,837,5739.

### Prophylactic and Therapeutic Utilities

One embodiment of the present invention is a method of growing cells and tissues that may be transplanted into an affected person for the treatment of diseases including but not limited to cardiac diseases or cardiac disorders, hereditary, or genetic diseases, neurological or neurodegenerative diseases, traumatic injuries and cancers. A cosmetic application of the present invention is skin grafts for hair replacement and/or other such applications.

Stem cells can be induced to differentiate into various types of tissues originating from all three germ layers (endoderm, mesoderm, and ectoderm) including but not limited to skin, hair, nervous tissue, pancreatic islet cells, bone, bone marrow, pituitary gland, liver, bladder, and other tissues having diagnostic or therapeutic utility in animals, including humans.

The present invention provides a method of treating a disorder or disease state by generating, suitable replacement cells, groups of cells, tissues or organs from isolated non-adherent mesenchymal stem cells.

In a preferred embodiment, the non-adherent mesenchymal stem cells are transferred or transplanted in a patient suffering from cardiac disease or disorders. Examples include, but not limited to: myocarditis, Coronary Heart Disease, angina, Acute Coronary Syndrome, Aortic Aneurysm and Dissection, arrhythmias, Cardiomyopathy, Congenital Heart Disease, congestive heart failure or chronic heart failure, pericarditis, and the like.

Transplantation of cellular products into a region of damaged myocardium, termed cellular cardiomyoplasty, is a new therapeutic modality designed to replace or repair necrotic, scarred, or dysfunctional myocardium. Ideally, graft cells should be readily available, easy to culture to ensure adequate quantities for transplantation, and able to survive in host myocardium, often a hostile environment of limited blood supply and immunorejection. Most importantly, transplantation of graft cells should improve cardiac function and prevent ventricular remodeling, (see, for example, the examples section which follows). To date, a number of candidate cells have been transplanted in experimental models, including fetal and neonatal cardiomyocytes, embryonic stem cell derived myocytes, tissue engineered contractile grafts, skeletal myoblasts, several cell types derived from adult bone marrow (BM), and cardiac precursors/stem cells resident within the heart itself. There has been substantial clinical development of the use of whole BM, skeletal myoblasts and BM derived mesenchymal stem cells (MSCs) in trials enrolling both post-infarction patients and patients with chronic ischemic left ventricular dysfunction.

The identification and culture expansion of cardiac stem cells (CSCs) would be useful for treatment of cardiac damage. Stem cells from other tissues are stimulated by growth factors (GFs) that control both proliferation and differentiation to mature functional cells. In the examples section which follows, the role of GFs on cardiac derived stem cells isolated based upon the expression of the tyrosine kinase receptor c-kit were identified. Cardiac derived c-kit⁺ cells have stem cell properties including the potential to regenerate cardiomyocytes. As stem cell factor is the ligand for c-kit, it was hypothesized that SCF would be a key factor in control of proliferation and differentiation of CSCs. These studies herein, demonstrated synergy of SCF with other growth factors secreted by cardiac derived stromal cells and provide the basis for an ex vivo culture system for expansion and differentiation of CSCs.

Other illustrative disorders and disease states include but are not limited to traumatic injury (e.g., post-trauma repair and reconstruction, for limb replacement, spinal cord injury, burns, and the like) and birth defects; pathological and malignant conditions of the cells, tissues, and organs (e.g., cancer); and degenerative and congenital diseases of the cells and tissues of the muscles (e.g., cystic fibrosis, muscular dystrophy, cardiac conditions), nerves (e.g., Alzheimer's, Parkinson's, and multiple sclerosis), epithelium (e.g., blindness and myopathy, atherosclerosis and other stenotic vascular conditions, enzyme deficiencies such as Crohn's disease, and hormone deficiencies such as diabetes), and connective tissues (e.g., immune conditions and anemia). Stem cells and tissues obtained from the methods described herein can be grafted or transplanted to a subject in need, preferably using the subject's own donor material.

The isolated stem cells of the present invention can also be differentiated into selected tissues by in vivo differentiation in immune compromised animal followed by isolation of the said tissues for a variety of therapeutic uses. The stem cells can also be cultured and differentiated in vitro for purposes of study, treatment or diagnostics.

In another preferred embodiment, the stem cells may be transformed with nucleic acids which code for different growth factors and/or cytokines which will aid in the differentiation of the stem cells if the organ of interest is damaged to the extent that the microenvironment is not supportive of cell differentiation.

In another preferred embodiment, the stem cells can be transformed with a ligand or receptor which will home a particular stem cell to the desired in vivo organ or tissue location, for example, heart.

The stem cells or stromal cells can be genetically-engineered using conventional techniques. The DNA encoding the desired ligand or receptor can be inserted into a vector and introduced unto the cells using techniques such as electroporation and/or retroviral infection. Other techniques which can be used to introduce DNA into the cells are calcium phosphate precipitation (Graham and van der Eb, Virology 52:456 (1973) and DEAE-dextran (Cullen et al., Nature 307:241 (1984)).

Examples of ligand-receptor binding pairs include transforming growth factor (TGF) and transforming growth factor receptor (TGFR) or EGF Receptor; (EGFR) epidermal growth factor (EGF) and EGFR; tumor necrosis factor-α (TNF-α) and tumor necrosis factor-receptor (TNFR); interferon and interferon receptor; platelet derived growth factor (PDGF) and PDGF receptor; transferrin and transferrin receptor; avidin and biotin or antibiotin; antibody and antigen pairs; interleukin and interleukin receptor (including types 3, 4 and 5); granulocyte-macrophage colony stimulating factor (GMCSF) and GMCSF receptor; macrophage colony stimulating factor (MCSF) and MCSF receptor; and granulocyte colony stimulating factor (G-CSF) and G-CSF receptor. Further, the ligand-binding pair can be a pair wherein the first member is naturally-occurring and the second member is provided using genetic-engineering techniques. For example, the stromal cells can be genetically-engineered by inserting DNA encoding sugar receptors and this will enhance the homing of the stem cells to the stromal cells based upon the naturally-occurring sugar molecules present in stem cells (Aizawa et al; Exp. Hematol. 16: 811-813 (1988).

The terms ligand and receptor are intended to encompass the entire ligand or receptor or portions thereof. Portions which can be used within this invention are those portions sufficient for binding to occur between the ligand and the receptor.

The cells can be administered by subcutaneous or other injection or intraveneously. In methods for treating a host afflicted with a bone marrow associated disease, a therapeutically effective amount of stem cells or stromal cells is that amount sufficient to significantly reduced or eliminate the symptoms or effects of a bone marrow associated disease. The therapeutically effective amount administered to a host will be determined on an individual basis and will be based, at least in part, on consideration of the individual's size, the severity of symptoms to be treated, and the results sought. Thus, a therapeutic effective amount can be determined by one of ordinary skill in the art of employing such practice in using no more than routine experimentation.

Mammals: Mammals that are useful according to the invention include any mammal (for example, human, mouse, rat, sheep, rabbit, goat, monkey, horse, hamster, pig or cow). A non-human mammal according to the invention is any mammal that is not a human, including but not limited to a mouse, rat, sheep, rabbit, goat, monkey, horse, hamster, pig or a cow.

### Dosage and Mode of Administration

By way of example, a patient in need of non-adherent mesenchymal stem cells as described herein can be treated as follows. Cells of the invention can be administered to the patient, preferably in a biologically compatible solution or a pharmaceutically acceptable delivery vehicle, by ingestion, injection, or any number of other methods. A preferred method is endoscopic retrograde injection. Another preferred method is injection or placement of the cells or directly into cardiac tissue. The dosages administered will vary from patient to patient; a “therapeutically effective dose” can be determined, for example but not limited to, by the level of enhancement of function. Monitoring levels of stem cell introduction, the level of expression of certain genes affected by such transfer, and/or the presence or levels of the encoded product will also enable one skilled in the art to select and adjust the dosages administered. Generally, a composition including stem cells will be administered in a single dose in the range of 10⁵-10⁸ cells per kg body weight, preferably in the range of 10⁶-10⁷ cells per kg body weight. This dosage may be repeated daily, weekly, monthly, yearly, or as considered appropriate by the treating physician. The invention provides that cell populations can also be removed from the patient or otherwise provided, expanded ex vivo, transduced with a plasmid containing a therapeutic gene if desired, and then reintroduced into the patient.

### Pharmaceutical Compositions

The invention provides for compositions comprising a stem cell according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

After pharmaceutical compositions comprising a compound of the invention formulated in an acceptable carrier have been prepared, they can be placed in an appropriate container and labeled for treatment of an indicated condition with information including amount, frequency and method of administration.

### General Methods for Isolation of Cells

Sources of Stem Cells: Except where otherwise required, the invention can be practiced using stem cells of any vertebrate species. Included are stem cells from humans; as well as non-human primates, domestic animals, livestock, and other non-human mammals.

Embiyonic Stem Cells: Embryonic stem cells can be isolated from blastocysts of members of the primate species (Thomson et al., Proc. Natl. Acad. Sci. USA 92:7844, 1995). Human embryonic stem (hES) cells can be prepared from human blastocyst cells using the techniques described by Thomson et al. (U.S. Pat. No. 5,843,780; Science 282:1145, 1998; Curr. Top. Dev. Biol. 38:133 ff., 1998) and Reubinoff et al, Nature Biotech. 18:399 (2000)).

Briefly, human blastocysts arc obtained from human in vivo preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology arc individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (about 200 U/mL; Gibco) or by selection of individual colonies by micropipette. Clump sizes of about 50 to 100 cells are optimal.

Antibodies are particularity useful for the preparation of substantially pure stem cells. By “substantially pure” herein is meant that at least about 50% of the cells present after sorting are stem cells, with at least about 70% preferred and at least about 90% preferred.

Appropriate markers or antigens for detecting bone marrow derived cells (BMDC) are polypeptides or nucleic acids not normally found in tissues outside of the bone marrow. Examples of such markers include, but are not limited to, Flk-1 (Swissprot: locus VGR2_HUMAN, accession P35968), Sca-1 (Swissprot: locus ICE3_HUMAN, accession P42574), Thy-1 (Swissprot: locus THY1_HUMAN, accession PO4216), Patched (Accession NP—000255.1 GI:4506247), CXCR (NP—003458.1 GI:4503175), survivin (Swissprot: locus BIR5_HUMAN, accession 015392), and the human homolog of mouse nucleostatin (NP—705775.1 GI:23956324) polypeptides and nucleic acids encoding all or a portion of these proteins. These polypeptides and nucleic acids can be readily obtained using methods well-known to those skilled in the art. Other BMDC markers can also be identified, for example, using transcriptional profiling techniques well-known to those skilled in the art, which can be used to determine the expression of specific subsets of genes in BMDC's and not in non-BMDC tissues. The further elucidation of BMDC-specific markers (e.g., associated with the bone-marrow stem-cell compartment and not historically associated with cancer) using the methods described herein, will allow the detection of BMDC associated metaplasias and cancers at the single level (e.g., by immunohistochemistry or nucleic acid amplification) prior to detection by conventional methods. Immunological based diagnostic and prognostic assays such as those described herein, utilize an antibody that is specific for a BMDC polypeptide (i.e., an antigen normally found only in BMDC's) which can be a polyclonal antibody or a monoclonal antibody and in a preferred embodiment is a labeled antibody.

In one preferred embodiment, the population of stem cells is purified. A purified population of stem cells contains a significantly higher proportion of stem cells than the crude population of cells from which the stem cells are isolated. For example, the purification procedure should lead at least to a five fold increase, preferably at least a ten fold increase, more preferably at least a fifteen fold increase, most preferably at least a twenty fold increase, and optimally at least a twenty-five fold increase in stem cells with respect to the total population. The purified population of stem cells should include at least 15%, preferably at least 20%, more preferably at least 25%, most preferably at least 35%, and optimally at least 50% of stem cells.

The purified population of stem cells may be isolated by contacting a crude mixture of cells containing a population of stem cells that express an antigen characteristic of stem cells with a molecule that binds specifically to the extracellular portion of the antigen. Such a technique is known as positive selection.

Procedures used to isolate stem cells are described in detail in the Examples which follow. However, isolation of cells useful in the present invention can be obtained by any method that is well known in the art. For example, bone marrow derived hematopoietic stem cells can be isolated by density gradient centrifugation, e.g., with Ficoll/Hypaque. Specific cell populations can be depleted or enriched using standard methods using stem cell-specific mAbs (e.g., anti-CD34 mAbs). Specific cell populations can also be isolated by fluorescence activated cell sorting according to standard methods. Monoclonal antibodies to cell-specific surface markers known in the art and many are commercially available. The binding of the stem cells to the molecule permit the stem cells to be sufficiently distinguished from contaminating cells that do not express the antigen to permit isolating the stem cells from the contaminating cells. For example, Lin⁻, Sca⁺, c-kit⁺, CD34⁺.

The molecule used to separate stem cells from the contaminating cells can be any molecule that binds specifically to the antigen that characterizes the stem cell. The molecule can be, for example, a monoclonal antibody, a fragment of a monoclonal antibody, or, in the case of an antigen that is a receptor, the ligand of that receptor. For example, VEGF. The number of antigens, such as VEGF receptors, characteristic of stem cells found on the surface of such cells, must be sufficient to isolate purified populations of such cells. For example, the number of antigens found on the surface of stem cells should be at least approximately 1,000, preferably at least approximately 5,000, more preferably at least approximately 10,000, most preferably at least approximately 25,000, and optimally at least approximately 100,000. There is no limit as to the number of antigens contained on the surface of the cells. For example, the cells may contain approximately 150,000, 250, 000, 500,000, 1,000,000, or even more antigens on the surface.

The source of stem cells may be any natural or non-natural mixture of cells that contains stem cells. The source may be derived from an embryonic mammal, or from the post-natal mammal. One source of cells is the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

Either before or after the crude cell populations are purified as described above, the population of stem cells may be further concentrated by methods known in the art. For example, the stem cells can be enriched by positive selection for one or more antigens characteristic of stem cells. Such antigens include, for example, FLK-1, CD34, and AC133. For example, human stem cells may be pre-purified or post-purified by means of an anti-CD34 antibody, such as the anti-My-10 monoclonal antibody described by Civin in U.S. Pat. No. 5,130,144. The hybridoma cell line that expresses the anti-My monoclonal antibody is available from the American Type Culture Collection, 12301 Parklawn Drive, Rockville, Md. 20852, USA. Some additional sources of antibodies capable of selecting CD34⁺ cells include AMAC, Westbrook, Me.; Coulter, Hialea, Fla.; and Becton Dickinson, Mountain View, Calif. CD34⁻ cells may also be isolated by means of comparable antibodies, which may be produced by methods known in the art, such as those described by Civin in U.S. Pat. No. 5,130,144.

In addition, or as an alternative to, the enrichment with anti-CD34 antibodies, populations of stem cells may also be further enriched with anti-Sca antibodies; with the AC 133 antibodies described by Yin et al., Blood 90, 5002-5112 (1997) and by Miraglia et al., Blood, 90, 50135021 (1997). The AC133 antibodies may be prepared in accordance with Yin et al.; ibid, or purchased from Miltenyi Biotec.

In accordance with the invention, stem cells can also be detected using for example, antibodies to c-kit. The c-kit proto-oncogene encodes a transmembrane tyrosine kinase receptor for an unidentified ligand and is a member of the colony stimulating factor-1 (CSF-1)—platelet-derived growth factor (PDGF)—kit receptor subfamily. c-kit was shown to be allelic with the white-spotting (W) locus of the mouse. Mutations at the W locus affect proliferation and/or migration and differentiation of germ cells, pigment cells and distinct cell populations of the hematopoietic system during development and in adult life. The effects on hematopoiesis are on the erythroid and mast cell lineages as well as on stem cells, resulting in a macrocytic anemia which is lethal for homozygotes of the most severe W alleles, and a complete absence of connective tissue and mucosal mast cells. W mutations exert their effects in a cell autonomous manner, and in agreement with this property, c-kit RNA transcripts were shown to be expressed in targets of W mutations (Nocka, K., Majumder, S., Chabot, B., Ray, P., Cervone, M., Bernstein, A. and Besmer, P. (1989) Genes & Dev. 3, 816-826.). High levels of c-kit RNA transcripts were found in primary bone marrow derived mast cells and mast cell lines. Somewhat lower levels were found in melanocytes and erythroid cell lines. The identification of the ligand for c-kit is of significance and interest because of the pleiotropic effects it might have on the different cell types which express c-kit and which are affected by W mutations in vivo. The demonstration of identity of c-kit with the W locus implies a function for the c-kit receptor system in various aspects of melanogenesis, gametogenesis and hematopoiesis during embryogenesis and in the adult animal.

The ligand of the c-kit receptor, KL, has been identified and characterized, based on the known function of c-kit/W in mast cells (Zsebo, K. M., et al., (1990a) Cell 63, 195-201; Zsebo, K. M., et al., Cell 63, 213-214 (1990B). The c-kit receptor in hematopoiesis KL stimulates the proliferation of bone marrow derived and connective tissue mast cells and in erythropoiesis, in combination with erythropoietin, KL promotes the formation of erythroid bursts (day 7-14 BFU-E). Furthermore, recent in vitro experiments with KL have demonstrated enhancement of the proliferation and differentiation of erythroid, myeloid and lymphoid progenitors when used in combination with crythropoietin, GM-CSF, G-CSF and IL-7 respectively suggesting that there is a role for the c-kit receptor system in progenitors of several hematopoietic cell lineages.

As used herein, c-kit ligand protein and polypeptide encompasses both naturally occurring and recombinant forms, i.e., non-naturally occurring forms of the protein and the polypeptide which are sufficiently identically to naturally occurring c-kit to allow possession of similar biological activity. Examples of such polypeptides includes the polypeptides designated KL-1.4 and S-KL, but are not limited to them. Such protein and polypeptides include derivatives and analogs. In one embodiment of this invention, the purified mammalian protein is a murine protein. In another embodiment of this invention, the purified mammalian protein is a human protein.

Cells may be further enriched for stem cells by removing cells that are Lin⁺. Such a method is known as negative selection. Negative selection may be used either before or after positive selection. Thus, molecules, such as antibodies or fragments of antibodies, that bind to all or any combination of CD1, CD2, CD3, CD4, CD5, CD8, CD10, CD11b, CD13, CD14, CD15, CD16, CD19, CD20, CD24, CD25, CD28, CD29, CD33, CD36, CD38, CD41, CD41a, CD56, CD66b, CD66e, CD69, and glycophorin A may be used to remove the unwanted Lin⁺ cells by the same methods described above for positive selection.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

The above disclosure generally describes the present invention. A more complete understanding can be obtained by reference to the following specific examples, which are provided herein for purposes of illustration only and are not intended to limit the scope of the invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Non-Adherent Mesenchymal Stem Cells

Materials and Methods

Human Bone Marrow Mononuclear Cells: Human BM cells were obtained from AllCells LLC (Emeryville, Calif.), the BM cells were collected from normal donors under appropriate informed consent and Institutional Board Review approved. The BM was shipped at room temperature overnight and the mononuclear fraction was isolated on a Ficol gradient.

The BM MNC cells were labeled with anti CD271 (low-affinity nerve growth factor receptor, LNGFR) (Miltenyi Biotec Inc., Auburn, Calif.) and the CD271⁻ cells isolated using a MACS cell separation device (Miltenyi Biotec Inc., Auburn, Calif.). The CD271⁺ cells were cultured in 100 ml Teflon bags (American Fluoroseal, Gaithersburg, Md.) in 50 ml of alpha MEM plus 20% FCS supplemented with 20 ng/ml bFGF (Peprotech Inc, Rocky Hill, N.J.).

Plastic adherent MSC (PA-MSC) were also generated from the BM MNC using standard culture conditions in T162 Corning (Acton, Mass.) tissue culture flasks at 1-5×10⁶ cell/ml in alpha MEM media containing 20% FCS (α-MEM+20% FCS). The cells were incubated in 5% CO₂, at 37° C. and the media changed weekly. Adherent cells grew in the cultures and were passaged using trypsin when confluent.

Induction and Assessment of Multilineage Differentiation Potential: Human bone marrow CD271⁺ cells were harvested from the culture bag and plated in 6-well cell culture dishes (Nunc, Roskilde, Denmark) for differentiation assays. Adipocytic and osteoblastic differentiation was induced according to the manufacture's protocol from Miltenyi Biotec Inc.

Briefly, adipocytic differentiation was induced by culturing these cells in NH AdipoDiff Medium (Miltenyi Biotec Inc., Auburn, Calif., USA) at a concentration of 5×10⁴ cells/ml for 2 weeks. Then cells were used for lipid droplet staining using Oil Red O (Sigma-Aldrich, St. Louis, Mo.). Osteogenic differentiation was induced by culturing these cells in NH OsteoDiff Medium (Miltenyi Biotec Inc.) at a concentration of 3×10⁴ cells/ml for 3 weeks. Then the cells were stained with SIGMA FAST BCIP/NBT Buffered Substrate Tablet (Sigma) to detect their expression of alkaline phosphatase (AP), an enzyme that is involved in the bone matrix mineralization. Cells that were cultured in alpha-MEM during this period were used as controls.

TABLE 1
Flow analysis of CD271⁺ cells

| Antibody | % |
|---|---|
| CD105 | 2% |
| CD45 | 86% |
| CD90 | 23% |
| CD73 | 12% |

Isolation of NA-MSC: Mesenchymal stem cell (MSC) precursors were isolated using magnetic cell selection with an antibody to CD271 (low-affinity nerve growth factor receptor, LNGFR) resulting in enrichment of cells with CFU-F potential. The CD271⁺ cells were placed in 100 ml Teflon culture bags in alpha MEM plus 20% FCS plus 20 ng/ml rhbFGF.

At regular intervals the bags were massaged to prevent adherence of cells to the surface of the bag. After 5 to 7 days of culture, clusters of cells were present and the cells continued to proliferate forming spheres of MSC. These non adherent MSC (NA-MSC) were cultured for 2 to 3 months. Phenotypic analysis demonstrated a distinct phenotype of NA-MSC compared to plastic adherent mesenchymal stem cells (PA-MSC), e.g. the NA-MSC lacked expression of CD105. When the NA-MSC were placed in plastic tissue culture flasks the NA-MSC attached to the surface of the flask and proliferated as typical PA-MSC and expressed CD 105. The NA-MSC were multi potential cells with the potential for osteogenic and adipocyte differentiation. Current studies are evaluating the potential of NA-MSC to integrate into damaged cardiac tissue in NOD/SCID mice.

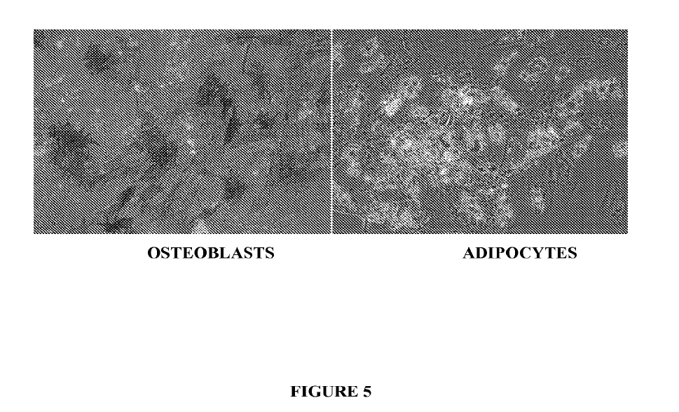

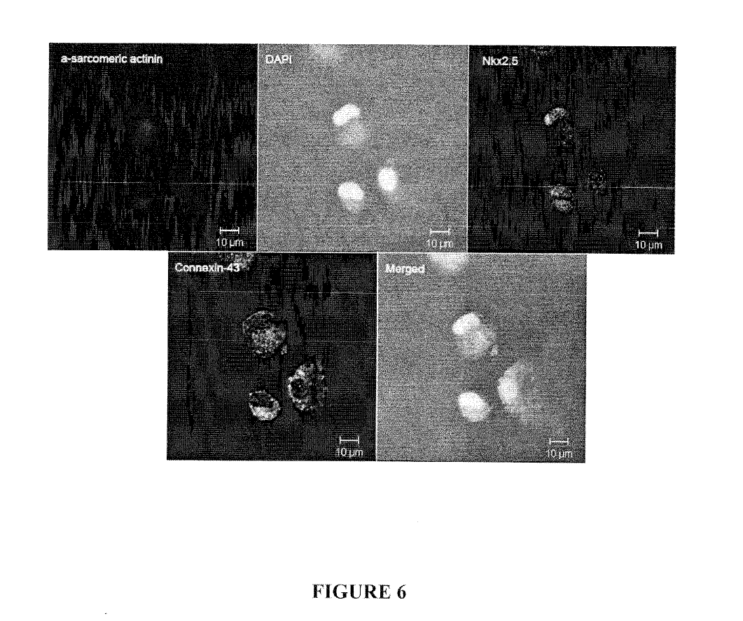

Differentiation of NA-MSC: The differentiation potential of non-adherent mesenchymal stem cells (NA-MSC) was evaluated using (i) standard conditions for osteoblast and adipocyte differentiation; and (ii) culture in a cocktail of growth factors as reported by Behfar & Terzic (Nature Clinical Practice 3 supl. 1, p S78). The NA-MSC formed ostcoblasts and adipocytes as shown in FIG. 5. When cultured in the cocktail of growth factors, NA-MSC demonstrated a cardiogenic potential with expression of alpha sarcomeric actinin and Nkx2.5 (FIG. 6).

Conclusion: The data presented in this study demonstrate the generation of non-adherent mesenchymal stem cells (NA-MSC) from CD271⁺ cells isolated from normal human BM. After culture for 3 to 4 weeks the NA-MSC express CD105, have the typical adherent morphology of MSC when cultured on plastic and can differentiate into multiple cell types including osteoblasts and adipocytes. Further the NA-MSC expressed cardiac makers after culture in cardiac inducing conditions. The differentiated cells also demonstrated beating typical of cardiac cells. Future studies will evaluate human NA-MSC in vivo in NOD/SCID mice following induction of MI.

### Example 2

### Expansion of c-kit⁺ Cardiac Stein Cells with in vivo Engraftment Potential in Non Adherent Culture Conditions

Cardiac Stem Cells (CSCs) were isolated based upon expression of c-kit. Using clonal assays in semi solid media, growth factor (GF) stimulation of c-kit+ cells isolated from human heart tissue were evaluated. Colonies were formed with different GFs and combinations of GFs with the largest colonies (diameter >1.0 mm) formed with the combination of stem cell factor (rhSCF) plus media conditioned (CM) by human heart stromal cells (HuHStr). These colonies contained cells with a primitive morphology that formed 20 colonies upon replating. Human c-kit⁺ cells were also grown in liquid culture in TEFLON bags with rhSCF and HuHStr CM. The cells proliferated over a two week period and formed spheres of c-kit⁺ cells that differentiated to a cardiac phenotype expressing Nkx2.5 and GATA-4. The cultured human c-kit⁺ cells (30,000/mouse) were also injected into the hearts of NOD/SCID mice following MI. 4-weeks after injection the mice were sacrificed and immunohistochemistry demonstrated extensive human myocytes (Alu⁺ cells) and human cells in vessel walls. In conclusion, these studies demonstrate a key role of SCF to stimulate CSCs in combination with other GFs to generate cells capable of engrafting ischemic cardiac tissue.

Materials and Methods

Isolation of c-kit+ CSCs from human heart tissue: Human fetal heart tissue was obtained with appropriate consent and IRB approval from aborted fetuses. The heart tissue was washed and dissected into small pieces and digested using collagenase for 5 minutes. The cell suspension was passed through a cell strainer and counted using Trypan Blue for viability.

In vitro culture of c-kit⁺ cells: a) Clonal Assay:

Human cardiac c-kit⁺ cells were assayed in semi solid media essentially as described for hematopoietic cells. An agarose layer (0.5%; 1 ml) was formed in 35 mm petri dishes with addition of growth factors (GFs) or conditioned media (CM). A second layer of methycellulose (MC) without added GFs, Stem Cell Technologies, Vancouver, Canada; 1 ml) containing c-kit⁺ cells was pipette over the agarose layer and the cultures incubated for 14 days at 37° C. in 5% CO₂. Colonies were scored using an inverted microscope with colonies defined by 50 or more cells.

b) Liquid Culture: Cardiac c-kit⁺ cells were cultured in Teflon bags (American Fluoroseal Inc, Gaithersburg, Md.) in alpha MEM plus 20% FCS supplemented with 100 ng/ml of recombinant human stem cells factor (rhSCF, Amgen Inc, Thousand Oaks, Calif.) and 10% media conditioned by human fetal heart stromal derived cells (HrtStr CM). The cultures were incubated at 37° C. in 5% CO₂ for 4 weeks with weekly media changes.

Injection of c-kit⁺ cells into infracted hearts of NOD/SCID mice: A myocardial infarction was induced in NOD/SCID mice by closure of the left anterior descending (LAD) coronary artery. On the day of infarction, while the chest was open for MI induction, the cells were injected directly into the myocardium. Multiple injections of 10 μl were delivered.

Echocardiography: Echocardiographic evaluation of cardiac anatomy was performed, under general anesthesia, at baseline, 1, 2 and 4 weeks post MI.

PV Loop: At the end of the study, mice were placed under general anesthesia, the carotid artery was cut-down and jugular vein access was obtained. A Millar SPR 839 catheter was progressed into the left ventricular (LV) and hemodynamic measurements of LV with and without occlusion in closed and open chest will be obtained. After PV measurement the heart was harvested and perfused with KCl and fixatives for immunohistochemical studies.

Results:

These experiments demonstrated synergy of stem cell factors (SCF) with other growth factors secreted by cardiac derived stromal cells and provide the basis for an ex vivo culture system for expansion and differentiation of cardiac stem cells (CSCs).

Clonal development of c-kit⁺ cells: Clonal assays which were developed for the study of hematopoietic stem and progenitor cells were adapted to evaluate the growth factor (GF) responsiveness of CSCs. Human fetal heart tissue was obtained, with appropriate institutional approvals, and the heart tissue digested with collagenase. A single cell suspension was prepared and labeled with an antibody to c-kit (CD117) conjugated to iron particles and the c-kit⁻ cells isolated using a Miltenyi VarioMACS selection device. The c-kit cells were plated in double layer semi solid cultures consisting of an underlay of 1 ml of 0.5% agar in alpha MEM plus 30% FCS and an overlay of lml of methylcellulose (Stem Cell Technologies Inc, Vancouver, Canada). GFs were incorporated into the underlay and the c-kit⁻ cells incorporated into the overlay. Cultures were incubated at 37° C. in 5% CO₂. Colonies could be visualized as early as 3 or 4 days of incubation and the number of cells in colonies increased through the entire incubation period of 14 days reaching thousands of cells per colony.

Colonies were scored on day 14 of culture and 10 cells were used as a minimal cell number to define a colony. As shown in Table 2, different GFs had differing effects on the c-kit⁺ cells.

TABLE 2
Colony formation of c-kit+ cells by GFs:

| Growth Factors | Concentration | | Number of Colonies |
|---|---|---|---|
| PBS | NA | | 0 |
| rh basic FGF | 20 | ng/ml | 0 |
| rhVEGF | 100 | ng/ml | 0 |
| rhSCF | 100 | ng/ml | 2 |
| rhEpo | 3 | U/ml | 1 |
| rhSCF + rh bFGF | | | 3 |
| rhSCF + rhVEGF | | | 2 |
| rhSCF + rhbFGF + rhVEGF | | | 1 |
| rhSCF + rhEpo | | | 4.5 |

Cultures contained 175,000 c-kit⁺ cells per 35 mm petri dish. Each GF or combination was plated in triplicate and the median numbers of colonies are presented. Abbreviations: rhSCF—recombinant human stem cell factor; FGF—fibroblast growth factor; VEGF—vascular endothelial growth factor; Epo—erythropoietin.

In separate experiments the potential of media conditioned by human cardiac derived stromal cells (HuHrtStr CM) was evaluated. Table 3 presents the colony numbers obtained. The maximal colony formation was obtained with rhSCF plus HuHrtStr CM. The combination of rhSCF plus rhEpo also resulted in increased colony numbers, however, the size of the colonies was smaller than the combination of rhSCF plus HuHrtStr CM and also had a red appearance which evidences the rhSCF plus rhEpo responsive cells may be erythroid precursors termed burst forming units erythroid (BFU-E). The number of cells in the colonies stimulated by rhSCF plus HuHrtStr CM was several thousand. To determine the proliferative potential of the cells within the colonies individual colonies were picked up from cultures of rhSCF plus HuHrtStr CM and replated the cells into secondary methylcellulose cultures. Cytospins were also prepared from colonies to evaluate the morphology of the cells which had a primitive blast appearance similar to the starting c-kit⁺ cells. Colony formation could be seen as early as 4 days of culture in the secondary cultures.

TABLE 3
Colony formation of c-kit⁺ cells by GFs:

| Growth Factors | Concentration | | Number of Colonies |
|---|---|---|---|
| PBS | NA | | 4 |
| rhSCF | 100 | ng/ml | 8 |
| rhEpo | 3 | U/ml | 8 |
| Hu Hrt Str CM (10X) | 100 | ul | 10 |
| rhSCF + rhEpo | | | 19 |
| rhSCF + Hu Hrt Str CM (10X) | | | 18 |

Cultures contained 50,000 c-kit⁺ cells per 35 mm petri dish. Each GF or combination was plated in triplicate and the median numbers of colonies are presented.

Up until the present study, human CSC populations have been limited to adherent cell populations that have been extensively passaged by continual trypsin treatment and recultured. The data presented above for clonal growth of human c-kit⁺ cells was undertaken with cultures containing an agar underlay to prevent exposure of the c-kit⁺ cells to plastic and the potential for adherence. In addition, identical cultures were established without an agar underlay and in addition to the colony formation described above, colonies of adherent cells formed in the cultures.

Liquid Culture of c-kit+ cells: Based upon the clonal data described above, c-kit⁺ cells were cultured in alpha MEM media plus 100 ng/ml rhSCF and 10% HuFHrtStr CM in 100 ml TEFLON bags with media change weekly. The cell numbers increased over time with clusters of cells developing from proliferating cells. In addition adherent cells formed on the surface of the Teflon bags, suggesting the presence of subpopulations of cells within the c-kit⁺ population. At weekly intervals the Teflon bags were massaged to release the adherent cells resulting in minimal adherent cells with time.

To evaluate the potential of the culture conditions to stimulate differentiation of the c-kit⁺ cells, cytospin slides were prepared of the cultured cells after 2 weeks and stained the slides for c-kit expression, for cardiac markers (GATA-4 and Nkx2.5) and for endothelial markers (VEGF receptor KDR). c-kit₊ cells were present in both cell clusters and as single cells at 2 weeks of culture. In addition, the cells demonstrated differentiation into both cardiac and endothelial cells lineages with positive straining for GATA-4, Nkx2.5 and KDR.

Injection of c-kit⁺ cells into NOD/SCID Mice: To evaluate the in vivo potential of the c-kit⁺ cells, cells which were cultured for 2 weeks in liquid culture of with rhSCF and HuFHrtStr CM, were injected into infracted heart tissue of NOD/SCID mice. For these experiments an infarct was induced by closure of the left anterior descending (LAD) coronary artery and while the chest was open for MI induction, the cells were injected directly into the myocardium. At 4 weeks post injection, the mice were sacrificed, hearts harvested and slides prepared. Staining for human cells in the mouse heart tissue using Alu specific probes demonstrated the presence of significant numbers of human cells. Significant numbers of human cells were observed within the heart tissue and in vascular structures. Functional parameters were also evaluated in these mice and as shown in FIG. 10 is the ejection fraction (EF) of mice injected with the c-kit+ cells or control mice injected with PBS. The infarct resulted in significant decreases in the EF of all mice with a greater decrease observed in the control mice.

Discussion:

In this report it the potential of c-kit⁻ cells, isolated from human fetal heart tissue was demonstrated to proliferate in semi solid media to form discrete colonies. The optimal colony formation is stimulated by the combination of rhSCF and media conditioned by human cardiac derived stromal cells. Based upon the colony formation data culture conditions for the human c-kit+ cells were developed that resulted in proliferation and differentiation. The same factors, rhSCF and HuFHrtStr CM stimulated proliferation of the c-kit⁺ cells in TEFLON bags over several weeks of culture. There was an expansion of c-kit⁺ cells and some cells differentiated to a cardiac phenotype with expression of Nkx2.5 and GATA-4.

It is proposed herein, that the culture of CSCs under non adherent conditions in TEFLON bags represents a more physiological condition compared to adherent growth on plastic. In addition, the non adherent conditions eliminate the need for enzyme treatment of the cells to detach from the plastic surface and minimize any surface antigen cleavage or modification that could occur with enzyme treatment. The conditions that have been defined herein are easily scalable for clinical trials and current studies are evaluating the expansion potential of the human c-kit⁺ cells.

The results generated by injection of the cultured human c-kit⁺ cells into NOD/SCID mice demonstrated the potential of these cultured cells for in vivo engraftment. Significant levels of human cells were observed in the mouse heart tissue at 4 weeks post injection of cultured c-kit⁺ cells. In addition, the data showed a trend to improved ejection fraction in the animals treated with cultured human c-kit⁺ cells compared to control animals. Considering the low dose of cells injected into each mouse (30,000 cells) the levels of human cells detected evidence expansion of the human cells in vivo. More extensive studies are currently being undertaken.

The repair of ischemic cardiac tissue offers improved health for many individuals who have suffered a heart attack. We have demonstrated in other studies that the injection of MSC into infracted heart tissue results in migration of endogenous CSCs to the ischemic area suggesting that MSC secrete GFs that stimulate migration and we hypothesize that the continued presence of the MSC is essential for further proliferation and differentiation of CSCs. The data presented in this study identifies SCF as a key GF involved in the control of CSCs. In addition these results demonstrate a stimulatory role of cardiac stromal cells through secreted GFs.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of isolating and culturing non-adherent stem cells comprising:
obtaining a biological sample from an animal;
isolating bone marrow from the biological sample;
isolating and culturing cells obtained from the bone marrow;
contacting the cells with antibody and separating the cells to isolate stem cells; and,
isolating and culturing non-adherent stem cells.

2. The method of claim 1, wherein the antibody is directed to a cell surface marker comprising CD271 and Stro-1.

3. The method of claim 1, wherein the stem cells are separated by magnetic or cell sorting means.

4. The method of claim 1, wherein the cells are cultured in a pliable tissue culture container and mechanical forces are applied to prevent adherence of the cells in the tissue culture container.

5. The method of claim 1, wherein the non-adherent stem cells are isolated by phenotype CD271⁺, CD105⁻.

6. The stem cells of claim 1, wherein the non-adherent stem cells are mesenchymal stem cell precursors.

7. The method of claim 1, wherein the animal is a human subject.

8. A method of repairing and regenerating tissue in an animal comprising:
isolating stem cells from bone marrow of an animal;
isolating, culturing, and expanding non-adherent stem cells; and,
transferring the non-adherent stem cells into the animal.

9. The method of claim 8, the isolated stem cells comprising at least one marker: c-kit⁺, CD271, CXCR4, CD 133, SCA-1, Tra-1-60, CD 44, CD 73, CD 90, CD 105 or Stro-1.

10. The method of claim 9, wherein the isolated stem cells have a CD271⁺, CD105⁻ phenotype.

11. The method of claim 8, wherein the isolated stem cells are optionally cultured in tissue culture comprising differentiation or growth factors.

12. The method of claim 8, wherein the stem cell recipient animal is also the donor of the bone marrow.

13. The method of claim 8, wherein the stem cells are obtained from allogeneic, autologous or syngeneic sources.

14. The method of claim 8, wherein the stem cells are non-adherent mesenchymal stem cells.

15. The method of claim 8, wherein the stem cells are transplanted into cardiac tissues.

16. A method of repairing and regenerating heart tissue in a patient comprising:
isolating stem cells from a donor;
isolating, culturing, and expanding non-adherent stem cells; and,
transferring the non-adherent stem cells into the patient.

17. The method of claim 16, wherein the stem cells are isolated from any compartment of a donor comprising bone marrow, tissue, organs, fluids or combinations thereof.

18. The method of claim 16, wherein the isolated stem cells have a CD271⁺, CD105⁻ phenotype.

19. The method of claim 16, wherein the isolated stem cells arc optionally cultured in tissue culture comprising differentiation or growth factors.

20. The method of claim 16, wherein the stem cell recipient patient is also the donor of the bone marrow.

21. The method of claim 16, wherein the stem cells are obtained from allogeneic, autologous, heterologous, syngeneic or combinations thereof

22. The method of claim 16, wherein the stem cells are non-adherent mesenchymal stem cells.

23. An isolated stem cell having at least one stem cell marker said marker comprising: CD271, CXCR4, CD 133, SCA-1, Tra-1-60, CD 44, CD 73, CD 90, CD 105 or Stro-1.

24. The isolated stem cell of claim 23, wherein the isolated stem cell comprising CD271⁺, CD105⁻ marker phenotype when the stem cell is isolated and prior to culturing and expansion.

25. A culture system comprising:
a cell culture chamber wherein said chamber prevents cells from adhering to the chamber surface.

26. The culture system of claim 25, wherein the cell culture chamber comprises an inner chamber surface comprising a cell non-adherent surface.

27. The culture system of claim 26, wherein the cell non-adherent surface comprises a polymer, polytetrafluoroethylene, or polytetrafluoroethene.

28. The culture system of claim 25, wherein the culture chamber is malleable.

29. A method of engrafting stem cells into a patient in need thereof, comprising:
isolating stem cells from a donor;
isolating, culturing, and expanding non-adherent stem cells; and,
engrafting the stem cells into the patient.

30. The method of claim 29, wherein the stem cells are cultured ex-vivo under conditions as non-adherent stem cells in the presence or absence of growth or differentiation factors.

31. The method of claim 29, wherein the donor cells are syngeneic, allogeneic, xenogeneic, autologous, heterologous, or combinations thereof.

32. The method of claim 29, wherein the stem cells are isolated from any compartment of a donor comprising bone marrow, tissue, organs, fluids or combinations thereof.

33. The method of claim 32, wherein the stem cells are cardiac stem cells.
